(19) United States
(12) Patent Application Publication
(10) Pub. No.: US 2015/0374544 A1
(43) Pub. Date: Dec. 31, 2015

(54) SURGICAL DEVICE HAVING A COMPLIANT TIP WITH A STEPPED OPENING

(71) Applicant: Synergetics, Inc., O'Fallon, MO (US)

(72) Inventor: Marc Fawzy George Estafanous, Orange, OH (US)

(21) Appl. No.: 14/750,315

(22) Filed: Jun. 25, 2015

Related U.S. Application Data
(60) Provisional application No. 62/016,738, filed on Jun. 25, 2014.

(51) Int. Cl. A61F 9/007; A61M 1/00; A61B 17/00

(52) U.S. Cl. CPC A61F 9/00727; A61B 17/00234; A61M 1/0084; A61B 2217/005; A61B 2217/007; A61M 2205/0216; A61M 2210/0612; A61M 2205/587

(57) ABSTRACT

A surgical device for use with a surgical site within a body includes a hub, a tube, and a compliant tip. The hub is configured to be connected to at least one of a vacuum source and an infusion source. The tube has a proximal end and an opposing distal end. The proximal end of the tube is connected to the hub. The compliant tip is connected to the tube and disposed adjacent the distal end of the tube, and includes a tip passage defined within the tip and a notch that defines a stepped opening in the tip. The stepped opening provides fluid communication between the at least one vacuum source and infusion source and the surgical site.

### CROSS-REFERENCE TO RELATED APPLICATIONS

This patent application claims priority to U.S. Provisional Patent Application No. 62/016,738, filed on Jun. 25, 2014, the disclosure of which is hereby incorporated by reference in its entirety.

### FIELD

This disclosure generally relates to surgical devices and, more specifically, to surgical devices for use in ophthalmic procedures.

### BACKGROUND

Surgical instruments used in ophthalmic surgical procedures typically include a cannula disposed at a distal end of the surgical instrument to perform various functions in connection with the ophthalmic surgical procedure. Some typical ophthalmic surgical procedures include vitrectomies, vitreoretinal procedures, and other procedures used to repair a detached retina in a human eye. In these procedures, a cannula is sometimes used to unfold a folded retina, to fill the vitreous cavity of the eye with air or gas, and to remove subretinal fluids (e.g., vitreous fluid) trapped behind the retina by attaching the cannula to a suction source to reattach the retina.

At least some known cannulas include a generally soft, compliant tip to prevent damaging delicate tissues within the eye during ophthalmic procedures. Conventional soft tip cannulas can become attached to tissue within the eye (e.g., the retina), particularly when suction is used to remove subretinal fluid through the cannula. For example, the opening at the end of known soft tip cannulas can become blocked or covered by tissue within the eye during an ophthalmic procedure. Suction applied to the cannula pulls this tissue into the opening, thereby attaching the cannula to the tissue. Such attachment can impede a user's ability to handle and maneuver the surgical instrument within the eye, as the cannula must be detached from the eye tissue (e.g., by reversing fluid flow through cannula) before moving the cannula to a different location within the eye. The attachment of the retinal tissue can also lead to tearing of the tissue and enlargement of existing breaks. Accordingly, a more satisfactory surgical device for use in ophthalmic procedures is needed.

This Background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

In one aspect, a surgical device for use with a surgical site within a body is provided. The surgical device includes a hub, a tube, and a compliant tip. The hub is configured to be connected to at least one of a vacuum source and an infusion source. The tube has a proximal end and an opposing distal end. The proximal end of the tube is connected to the hub. The compliant tip is connected to the tube and disposed adjacent the distal end of the tube, and includes a tip passage defined within the tip and a notch that defines a stepped opening in the tip. The stepped opening provides fluid communication between the at least one vacuum source and infusion source and the surgical site.

In another aspect, a surgical device for use with a surgical site within a body is provided. The surgical device has a proximal end and an opposing distal end. The proximal end of the surgical device is configured to be coupled to at least one of a vacuum source and an infusion source. The surgical device includes a tube and a compliant tip. The tube has a proximal end, an opposing distal end, and an internal tube passage defined therebetween. The compliant tip is connected to the tube and disposed adjacent the distal end of the tube, and has a distal end that extends beyond the distal end of the tube. The tip includes a tip passage defined therein and a notch defining a stepped opening in the tip. The stepped opening provides fluid communication between the at least one vacuum source and infusion source and the surgical site.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

FIG. 1 is a side view of an example surgical device of the present disclosure;

FIG. 2 is an enlarged cross-section of a portion of the surgical device shown in FIG. 1 indicated by line “2-2” in FIG. 1;

FIG. 3 is an end view of the distal end of the surgical device shown in FIG. 2;

FIG. 4 is a partial top view of the surgical device shown in FIG. 2; and

FIG. 5 is a cross-section of the portion of the surgical device shown in FIG. 2 including an illumination device.

### DETAILED DESCRIPTION

This disclosure generally relates to surgical devices for use in surgical sites within a body. For example, the surgical device of one embodiment is particularly suitable for use in ophthalmic procedures. As described in more detail herein, surgical devices of the present disclosure include a notch defining a stepped opening at a distal end of the device. The configuration of the surgical devices described herein minimize damage to delicate tissue within the body (for example, tissue within the eye), and also facilitate handling the surgical device during surgical procedures.

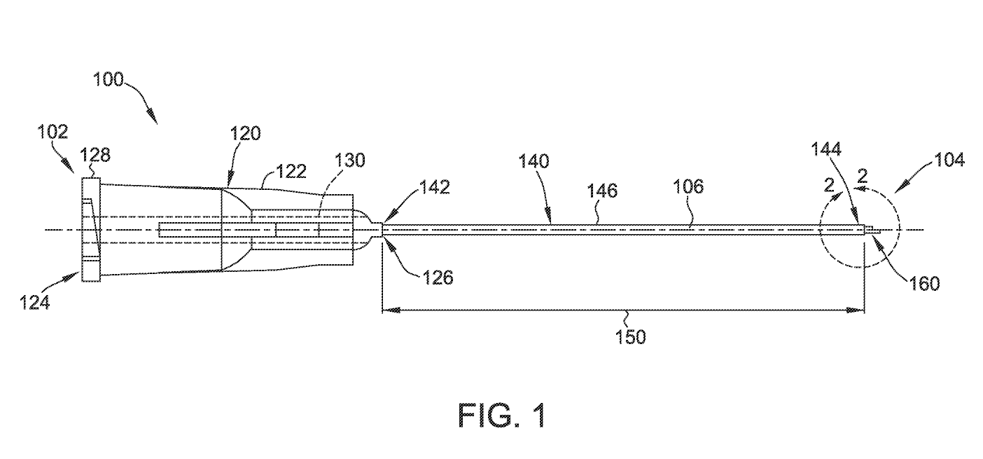

Referring to FIG. 1, an example surgical device of the present disclosure is indicated generally at 100. The surgical device 100 has a proximal end 102 and a distal end 104 configured to be inserted into a surgical site within a body. In the example embodiment, surgical device 100 generally includes a hub 120 disposed at proximal end 102 of surgical device 100, a cylindrical tube 140 connected to hub 120 and extending towards distal end 104, and a compliant tip 160 disposed at distal end 104 of surgical device 100. The illustrated surgical device 100 is particularly suitable for use in ophthalmic procedures. More specifically, in use, hub 120 is connected to a vacuum source and/or an infusion source, tube 140 is inserted into an incision in an eye (such as through a cannula positioned in the sclera that provides access to the surgical site), and compliant tip 160 is used to provide suction and/or to introduce fluids or gases at a surgical site within the eye. Although surgical device 100 is described herein as being suitable for use in ophthalmic procedures, surgical device 100 may be used in surgical procedures other than ophthalmic procedures.

In the example embodiment, hub 120 has a generally cylindrical body 122 having a proximal end 124 and a distal end 126. Hub 120 includes a connector 128 disposed at proximal end 124. In the example embodiment, connector 128 is configured to be connected to a vacuum source (not shown), such as, for example, a vacuum pump. Connector 128 may additionally or alternatively be configured to be connected to an infusion source, such as, for example, a pressurized gas or fluid source. Further, connector 128 may be configured to be connected to a handle (not shown) to facilitate handling of surgical device 100 during surgical procedures. Alternatively, hub 120 and/or tubing connected to hub 120 may be used as a handle during surgical procedures.

Hub 120 also includes an internal hub passage 130 defined within body 122. Hub passage 130 extends from proximal end 124 of hub 120 to distal end 126 of hub 120, and provides fluid communication between proximal end 124 of hub 120 and tube 140.

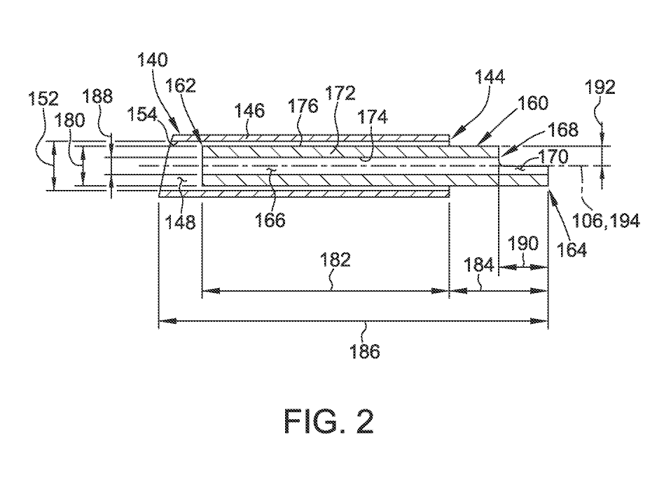

Tube 140 has a proximal end 142 and an opposing distal end 144, and includes a generally rigid, cylindrical tube sidewall 146 extending from proximal end 142 of tube 140 to distal end 144 of tube 140. Further, tube 140 includes an internal tube passage 148 (FIG. 2) extending from proximal end 142 of tube 140 to distal end 144 of tube 140.

Proximal end 142 of tube 140 is connected to distal end 126 of hub 120 such that tube passage 148 is in fluid communication with hub passage 130. Tube 140 extends from distal end 126 of hub 120 a length 150 along a longitudinal axis 106 of surgical device 100 to distal end 144 of tube 140. Tube 140 extends a suitable length 150 to enable posterior regions of the eye (e.g., the retina) to be accessed with surgical device 100 during surgical procedures. In the example embodiment, length 150 of tube 140 is between about 1.0 inches and 2.0 inches and, more suitably, between about 1.0 inches and about 1.5 inches. Further, in the example embodiment, tube 140 is substantially parallel to longitudinal axis 106 along the entire length 150 of tube 140. In alternative embodiments, tube 140 may be bent or curved to provide access to portions of the eye that are otherwise difficult to reach with a straight tube. Further, in alternative embodiments, tube 140 may have any suitable length that enables surgical device 100 to function as described herein.

Tube sidewall 146 is fabricated from a generally lightweight, rigid material. Suitable rigid materials from which tube sidewall 146 may be fabricated include, for example, hard plastics and stainless steel. Alternatively, tube sidewall 146 may be fabricated from any suitable material that enables surgical device 100 to function as described herein.

As shown in FIG. 2, tube passage 148 has a diameter 152 defined by an inner circumferential surface 154 of tube sidewall 146. In the example embodiment, diameter 152 of tube passage 148 is between about 0.010 inches and about 0.040 inches and, more suitably, between about 0.020 inches and about 0.030 inches. In alternative embodiments, tube passage 148 may have any suitable diameter that enables surgical device 100 to function as described herein.

Compliant tip 160 has a proximal end 162 and a distal end 164, and includes a tip passage 166 extending from proximal end 162 of compliant tip 160 to distal end 164 of compliant tip 160. Compliant tip 160 is connected to tube 140 such that tip passage 166 is in fluid communication with tube passage 148. In the illustrated embodiment, compliant tip 160 is connected to distal end 144 of tube 140, although in other embodiments compliant tip 160 may be connected to any suitable portion of tube 140 (e.g., proximal end 142) that enables surgical device 100 to function as described herein.

Compliant tip 160 includes a notch 168 defining a stepped opening 170 in compliant tip 160 that provides fluid communication between tip passage 166 and a surgical site in which surgical device 100 is used. Moreover, compliant tip 160 is fabricated from a generally soft, resilient, and flexible material to minimize trauma to delicate tissues within the eye during ophthalmic procedures. Suitable materials from which compliant tip 160 may be fabricated include, for example, silicone rubber and polyurethane. As described in more detail herein, the configuration of compliant tip 160 facilitates minimizing damage to delicate tissues within the eye, and also facilitates handling of surgical device 100 during surgical procedures.

In the example embodiment, compliant tip 160 includes a circumferential tip sidewall 172 extending from proximal end 162 of compliant tip 160 to distal end 164 of compliant tip 160. Tip sidewall 172 has an inner circumferential surface 174 and an outer circumferential surface 176. Compliant tip 160 has an outer diameter 180 defined by outer circumferential surface 176 of tip sidewall 172. As shown in FIG. 2, outer diameter 180 is sized to be received within tube 140. In the example embodiment, compliant tip 160 is connected to tube 140 by an interference fit. Additionally and/or alternatively, compliant tip 160 is connected to tube 140 by an adhesive, by a threaded connection, or by any other suitable connection that enables surgical device to function as described herein. The spacing between outer circumferential surface 176 of tip sidewall 172 and inner circumferential surface 154 of tube sidewall 146 is exaggerated in FIGS. 2-4 for purposes of illustration.

As shown in FIG. 2, when compliant tip 160 is connected to tube 140, a first length 182 of compliant tip 160 is disposed within tube 140, and a second length 184 of compliant tip 160 protrudes longitudinally outward from distal end 144 of tube 140. In the example embodiment, first length 182 is between about 0.05 inches and about 0.20 inches, and, more suitably, between about 0.05 inches and about 0.15 inches. Further, in the example embodiment, second length 184 is between about 0.01 inches and about 0.08 inches and, more suitably, between about 0.02 inches and about 0.06 inches. Further, in the example embodiment, compliant tip 160 has an overall length 186 of between about 0.06 inches and about 0.24 inches and, more suitably, between about 0.10 inches and about 0.20 inches. In other suitable embodiments, first length 182, second length 184, and overall length 186 of compliant tip 160 may be any suitable length that enables surgical device 100 to function as described herein. In yet other suitable embodiments, tip 160 may be connected to the outside of tube sidewall 146 at distal end 144 of tube 140. That is, tube 140 may be disposed within tip passage 166, and distal end 164 of compliant tip 160 may extend beyond distal end 144 of tube 140.

As noted above, tip passage 166 extends from proximal end 162 of compliant tip 160 to distal end 164 of compliant tip 160. Tip passage 166 has an inner diameter 188 defined by inner circumferential surface 174 of tip sidewall 172.

Notch 168 extends inward from distal end 164 of compliant tip 160, and extends radially inward from tip sidewall 172. More specifically, notch 168 extends longitudinally inward from distal end 164 a length 190, and extends radially inward from outer circumferential surface 176 of tip sidewall 172 to a depth 192. Length 190 and depth 192 of notch 168 are sized such that stepped opening 170 defined by notch 168 provides suitable fluid flow between tip passage 166 and the surgical site in which surgical device 100 is used. In the example embodiment, length 190 of notch 168 is equal to about one half of second length 184 of compliant tip 160, and depth 192 of notch 168 is equal to about one half of outer diameter 180 of compliant tip 160. As a result, notch 168 extends radially inward from sidewall 172 to a longitudinal centerline 194 of compliant tip 160, which coincides with longitudinal axis 106 of surgical device 100 in the illustrated embodiment. In other suitable embodiments, notch 168 may have any suitable depth and length that enables surgical device 100 to function as described herein. In some suitable embodiments, for example, the ratio of length 190 of notch 168 to second length 184 of compliant tip 160 is between about 0.25 and about 0.75. In yet other suitable embodiments, the ratio of depth 192 to outer diameter 180 is between about 0.25 and about 0.75.

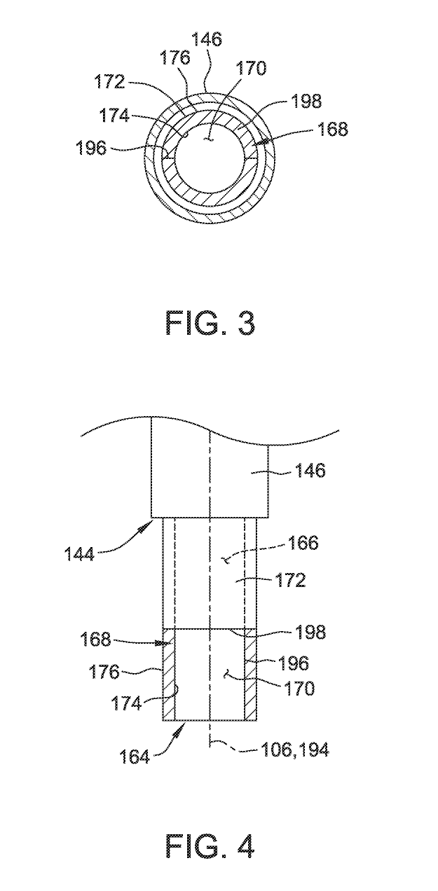

Referring to FIGS. 3-4, notch 168 is formed along a first surface 196 of tip sidewall 172 and a second surface 198 of tip sidewall 172. First surface 196 and second surface 198 extend between inner circumferential surface 174 of tip sidewall 172 and outer circumferential surface 176 of tip sidewall 172. Further, first surface 196 extends in a direction generally parallel to longitudinal centerline 194 of compliant tip 160, and second surface 198 extends along a circumferential direction of tip sidewall 172. In the example embodiment, first surface 196 of tip sidewall 172 is oriented substantially parallel to longitudinal centerline of 194 of compliant tip 160, and second surface 198 of tip sidewall 172 is oriented substantially perpendicular to longitudinal centerline 194 of compliant tip 160. Further, in the example embodiment, first surface 196 and second surface 198 are oriented substantially perpendicular to one another. In other suitable embodiments, first surface 196 may be oriented other than substantially parallel to longitudinal centerline 194 of compliant tip 160, second surface 198 may be oriented other than substantially perpendicular to longitudinal centerline 194 of compliant tip 160, and/or first surface 196 and second surface 198 may be oriented other than substantially perpendicular to one another. In some suitable embodiments, for example, first surface 196 and second surface 198 may be oriented at an angle of between about 90° and about 135° with respect to one another. In another suitable embodiment, first surface 196 and second surface 198 are curved surfaces, and together define a continuous, curved surface having a generally concave shape.

The surfaces 196, 198 along which notch 168 is formed define stepped opening 170. More specifically, as shown in FIGS. 3 and 4, stepped opening 170 is defined by first surface 196 and second surface 198. Because first surface 196 and second surface 198 are angled with respect to one another, opening 170 has a “stepped” configuration. In the illustrated embodiment, first surface 196 and second surface 198 adjoin one another along a discrete edge at an approximately 90 degree angle. In other suitable embodiments, first surface 196 and second surface 198 may be connected to one another by a generally curved or contoured surface, rather than a discrete edge, while still defining an opening having a stepped configuration.

Referring again to FIG. 2, compliant tip 160 includes a distal portion extending along length 190 (i.e., a “notched” portion) and a proximal portion extending from distal end 144 of tube 140 to the beginning of notch 168 (i.e., an “unnotched” portion). Because a portion of sidewall 172 is removed along the distal portion of compliant tip 160, the distal portion has a stiffness less than a stiffness of the proximal portion of compliant tip 160. As a result, the proximal portion of compliant tip 160 can be used to scrape and remove relatively stiffer membranes (e.g., within an eye) during surgical procedures.

The configuration of notch 168 and stepped opening 170 facilitates minimizing damage to delicate tissues within the eye, and also facilitates handling of surgical device 100 during surgical procedures by reducing the likelihood that opening 170 becomes blocked or covered by human tissue during surgical procedures. In particular, notch 168 and stepped opening 170 are shaped such that tissue within the eye is less likely to block or cover opening 170. As a result, the surgical devices of the present disclosure are less likely to become attached to human tissue during surgical procedures, even where suction is used to remove bodily fluids (e.g., vitreous or subretinal fluids). These surgical devices thereby minimize damage to delicate tissues within the eye and facilitate handling of the surgical device during surgical procedures. Further, the configuration of notch 168 in compliant tip 160 facilitates removal of relatively stiff membranes during surgical procedures as compared to traditional soft tip cannulas. In particular, notch 168 provides compliant tip 160 with varying degrees of stiffness along the length of compliant tip 160 such that the proximal portion of compliant tip 160 is relatively stiffer than the distal portion of compliant tip 160. That is, the notched portion of compliant tip 160 (i.e., the distal portion) is less stiff than the proximal portion as a result of a portion of the sidewall being removed. The distal portion thus exerts less of a bending force on the proximal portion, and the proximal portion behaves as a relatively stiff member that can be used to scrape and remove relatively stiff membranes.

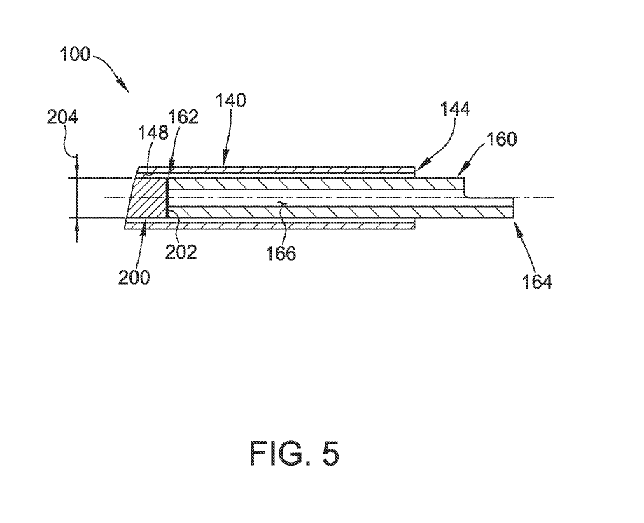

In some embodiments, surgical device 100 may also include an illumination device, such as fiber optic lights or lasers, configured to illuminate a surgical site. FIG. 5 is a cross-section of the portion of the surgical device 100 shown in FIG. 2 including an illumination device shown in the form of an optic fiber 200. Optic fiber 200 is configured to be connected to a light source (not shown), and to transmit light from the light source to a distal end 202 of optic fiber 200. Suitable materials from which optic fiber 200 may be constructed include, for example and without limitation, glass (e.g., silica glass) and plastic.

As shown in FIG. 5, optic fiber 200 is disposed within internal tube passage 148 defined by cylindrical tube 140, and distal end 202 of optic fiber 200 is disposed adjacent proximal end 162 of compliant tip 160. In some embodiments, distal end 202 of optic fiber 200 abuts proximal end 162 of compliant tip 160. In use, light transmitted through optic fiber 200 projects from distal end 202 of optic fiber 200, through tip passage 166 and/or around compliant tip 160, and into a surgical site (not shown) to illuminate the surgical site.

In the embodiment illustrated in FIG. 5, optic fiber 200 has a diameter 204 less than diameter 152 of tube passage 148 (shown in FIG. 2), and greater than outer diameter 180 of compliant tip 160 (shown in FIG. 2) such that optic fiber 200 fits within tube passage 148, but is obstructed from moving longitudinally beyond proximal end 162 of compliant tip 160. In other embodiments, diameter 204 of optic fiber 200 may be less than inner diameter 188 of tip passage 166 (shown in FIG. 2) such that optic fiber 200 fits within tip passage 166. In such embodiments, distal end 202 of optic fiber 200 may be disposed adjacent distal end 144 of tube 140 or distal end 164 of compliant tip 160.

When introducing elements of the present invention or the embodiments thereof, the articles “a”, “an”, “the” and “said” are intended to mean that there are one or more of the elements. The terms “comprising”, “including” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the invention, it is intended that all matter contained in the above description and shown in the accompanying figures shall be interpreted as illustrative and not in a limiting sense.

What is claimed is:

1. A surgical device for use with a surgical site within a body, the surgical device comprising:
a hub configured to be connected to at least one of a vacuum source and an infusion source;
a tube having a proximal end and an opposing distal end, the proximal end of the tube connected to the hub; and
a compliant tip connected to the tube and disposed adjacent the distal end of the tube, the tip including a tip passage defined therein and a notch defining a stepped opening in the tip, the stepped opening providing fluid communication between the at least one vacuum source and infusion source and the surgical site.

2. The surgical device of claim 1, wherein the tip has an outer diameter defined by a circumferential sidewall of the tip, the notch extending radially inward from the circumferential sidewall to a depth, wherein a ratio between the depth and the outer diameter is between about 0.25 and about 0.75.

3. The surgical device of claim 1, wherein the tip has a first length protruding longitudinally outward from the distal end of the tube, and the notch has a second length extending from a distal end of the tip towards a proximal end of the tip, wherein the ratio of the second length to the first length is between about 0.25 and about 0.75.

4. The surgical device of claim 1, wherein the tip includes a circumferential sidewall extending from a proximal end of the tip to a distal end of the tip, the tip passage defined within the circumferential sidewall, the notch defined by a first surface of the sidewall and a second surface of the sidewall, wherein the first surface is oriented at an angle of between about 90 degrees and about 135 degrees with respect to the second surface.

5. The surgical device of claim 4, wherein the first surface is oriented substantially perpendicular to the second surface.

6. The surgical device of claim 4, wherein the circumferential sidewall extends along a longitudinal direction of the tip, one of the first and second surfaces being oriented substantially perpendicular to the longitudinal direction.

7. The surgical device of claim 1, wherein the tip has an outer diameter sized to be received within the distal end of the tube.

8. The surgical device of claim 1, wherein the tip is fabricated from at least one of silicone rubber and polyurethane.

9. The surgical device of claim 1, wherein the tip is connected to the tube by an interference fit.

10. The surgical device of claim 1, wherein a distal portion of the tip has a first stiffness, and a proximal portion of the tip has a second stiffness, wherein the first stiffness is less than the second stiffness.

11. The surgical device of claim 1, further comprising an illumination device disposed within at least one of the tube and the compliant tip, the illumination device configured to transmit light from a light source to the surgical site.

12. A surgical device for use with a surgical site within a body, the surgical device having a proximal end and an opposing distal end, the proximal end configured to be connected to at least one of a vacuum source and an infusion source, the surgical device comprising:
a tube having a proximal end, an opposing distal end, and an internal tube passage defined therebetween; and
a compliant tip connected to the tube and disposed adjacent the distal end of the tube, the tip having a distal end that extends beyond the distal end of the tube, the tip including a tip passage defined therein and a notch defining a stepped opening in the tip, the stepped opening providing fluid communication between the at least one vacuum source and infusion source and the surgical site.

13. The surgical device of claim 12, wherein the tip is disposed within the internal tube passage, and the distal end of the tip protrudes outward from the internal tube passage.

14. The surgical device of claim 12, wherein the tip has an outer diameter defined by a circumferential sidewall of the tip, the notch extending radially inward from the circumferential sidewall to a depth, wherein a ratio between the depth and the outer diameter is between about 0.25 and about 0.75.

15. The surgical device of claim 12, wherein the tip has a first length protruding longitudinally outward from the distal end of the tube, and the notch has a second length extending from the distal end of the tip towards a proximal end of the tip, wherein the ratio of the second length to the first length is between about 0.25 and about 0.75.

16. The surgical device of claim 12, wherein the tip includes a circumferential sidewall extending from a proximal end of the tip to the distal end of the tip, the tip passage defined within the circumferential sidewall, the notch defined by a first surface of the sidewall and a second surface of the sidewall, the first and second surfaces extending between an outer circumferential surface of the sidewall and an inner circumferential surface of the sidewall.

17. The surgical device of claim 16, wherein the circumferential sidewall extends along a longitudinal direction of the tip, the first surface extending along the longitudinal direction of the tip and the second surface extending along a circumferential direction of the sidewall.

18. The surgical device of claim 17, wherein the first surface is oriented at an angle of between about 90 degrees and about 135 degrees with respect to the second surface.

19. The surgical device of claim 18, wherein the first surface is oriented substantially perpendicular to the second surface.

20. The surgical device of claim 12, wherein a distal portion of the tip has a first stiffness, and a proximal portion of the tip has a second stiffness, wherein the first stiffness is less than the second stiffness.
